(19) United States
(12) Patent Application Publication
(10) Pub. No.: US 2008/0031831 A1
(43) Pub. Date: Feb. 7, 2008

(54) Oral anti-calculus compositions and methods of use thereof

(75) Inventor: Moein Laali, Altenberge (DE)

Correspondence Address: LAW OFFICES OF KHALILIAN SIRA, LLC, 9100 PERSIMMON TREE ROAD, POTOMAC, MD, 20854, US

(73) Assignee: Technology & Marketing Consulting, Graz (AT)

(21) Appl. No.: 11/255,248

(22) Filed: Oct. 21, 2005

(51) Int. Cl. A61K 8/97; A61P 43/00

(52) U.S. Cl. 424/58

(57) ABSTRACT

The present invention provides anticalculus oral compositions containing a plant oil mixture comprising a major oil constituent and a minor oil constituent in a proportion of at least 10/1 respectively. The major oil constituent comprises an edible vegetable oil and the minor oil consitutent comprises emulsifying oil, flavoring oil, antiseptic oil, anti-inflammatory oil, or a combination thereof.

### FIELD OF THE INVENTION

The present invention relates to anticalculus and antiplaque compositions. In particular, the invention relates to oil-based compositions formulated for use as oral hygiene products.

### BACKGROUND OF THE INVENTION

Tooth decay and dental disease can be caused by bacterial action resulting from plaque formation around the teeth and/or the entrapment of food particles in interstices between teeth. As a result, decreasing the number of bacteria in the mouth has long been the target of dental care professionals.

Gum disease and oral health problems have reached epidemic proportions. According to the American Dental Association, three out of four adults over the age of 35 have some form of gum disease. Recently, the Surgeon General issued a report on oral health.

Studies have shown that gum (periodontal) disease not only can cause the loss of teeth, but can cause much more serious diseases. Individuals with gum disease have been shown to have an increased incidence of heart disease, diabetes, and respiratory disease and have increased risk of stroke, increased buildup of plaque in carotid arteries, weakened immune systems and increased risk of complications from diabetes. Treatment of gum disease by a periodontist usually is scaling and planning to remove hidden plaque and tartar below the gum line. In addition, in severe cases of gum disease, the diseased portions of the gums are surgically removed one quadrant at a time, and can potentially require the removal of teeth. These procedures are painful, expensive and merely treat the symptoms of the disease.

Oral plaque is responsible for both dental caries and periodontal disease, both of which remain quite pervasive in the human population. Plaque is a mucopolysaccharide which forms naturally in the mouth, as a result of the presence of saliva and bacteria. At the outset of plaque formation, plaque exists as a web of soft, sticky gelatin, called the pellicle. The pellicle provides a haven for the lodging and multiplication of bacteria. Thus, within a very short time, plaque becomes predominantly bacterial.

Microbial plaque develops on teeth and gums due to non-proper tooth brushing. As the time passes it combines with other materials and hardens into a rough, porous deposit of calcium carbonate (CaCO₃) or other insoluble carbonates which are the permanent and temporary hardness factors. Microbial plaque deposits between teeth and gums, mostly behind teeth of lower jaw from 3 to 3 which are constantly exposed to salvia enzyme, ptyalin and make a coarse and irritating feeling, causing gums to recede, and ultimately the teeth can become loose and fall out-or may have to be removed.

To remove the germs and plaque dentists use grinding tools. These tools harm tooth enamel and injure the gum, cause bleeding and systemic diseases, such as AIDS or Hepatitis. Additionally, scratches on enamel by grinding tool cause teeth to become sensitive to cold and touch.

The most common way of minimizing the number of bacteria is to brush and floss the teeth regularly, and to visit a dental hygienist to have the teeth and gums cleaned thoroughly. Another prior approach to control bacteria in the mouth is to rinse with a solution containing anti-microbial agents such as chlorhexidine digluconate. Removal of plaque and entrapped food particles reduces the incidence of caries, gingivitis, and mouth odors as well as generally improving oral hygiene.

Brushing has been found to be inadequate for removing all entrapped food particles and plaque. Therefore, to supplement brushing, dental flosses and tapes are often employed. After a toothbrush has been used, there are a large number of bacteria clinging to the toothbrush, even after the brush has been rinsed. This, of course, is undesirable, and these bacteria typically will multiply on the bristles between uses. To counter this problem, various approaches to making self-sterilizing toothbrushes have been described in the art including the use of antimicrobial agents.

U.S. Pat. No. 5,276,935 discloses a dental brush containing a plurality of parallel fibers having a medicament such as tetracycline, chlorhexidine, or sodium fluoride disposed between or within the individual fibers. The medicament is released during brushing to provide direct contact of the medicament with the areas in the mouth receiving treatment. One of the major side effects of rinsing the mouth or the toothbrush with anti-microbial agents such as chlorhexidine solution is the development of a yellow-brown stain on teeth, tongue, and fillings. Although this stain can be professionally removed, it is not cosmetically pleasing. In addition to the staining, taste disturbances in the perception of sweet and salt may develop, as well as scaling and soreness of the oral mucosa.

Many substances have been used individually and in combinations to improve gum and oral health. It would be highly desirable to combine the substances for optimal improvement of gum and oral health. A variety of compositions have been developed to attack the bacterial portion of plaque. For example, U.S. Pat. No. 4,335,022 describes a hydrous, enzymatic dental treatment composition used to destroy certain types of cariogenic bacteria via hydrolysis. However, enzymes are known to become unstable and to lose activity in the presence of water or moisture, rendering such hydrous enzymatic compositions both unstable and ineffective.

U.S. Pat. No. 4,515,772 describes an oral hygiene composition in the form of a mouthwash, liquid dentifrice or toothpaste which comprises an anionic anticalculus agent in the form of a water-soluble alkali-metal pyrophosphate.

Anhydrous toothpastes including an oil component and enzymatic additives are known, as exemplified by U.S. Pat. No. 3,574,824. Among the disadvantages of such prior known anhydrous oil-based toothpastes are an unpleasant mouth feel or oily sensation when used, and a reduced shelf life as compared to conventional, hydrous toothpastes due to the tendency of the oil component to oxidize and to become rancid. Additionally, these toothpastes are not optimal anti-plaque compositions, as the enzyme additive is rapidly diluted in the mouth and thus, is washed away from the tooth or gum area before it can be effective in fighting oral plaque.

U.S. Pat. No. 4,411,885 discloses an encapsulated or tableted oil-based dentifrice, including a source of vitamin E. The vitamin E component of the dentifrice is present in a quantity sufficient to reduce or to eliminate the unpleasant mouth feel or oily sensation associated with the previously known anhydrous oil-based toothpastes. The vitamin E also tends to reduce oxidation of the oil base and thus, to improve the shelf life of the dentifrice. While this encapsulated or tableted dentifrice eliminates many of the disadvantages of earlier dentifrices, it employs an abrasive agent, rather than an enzyme additive, to fight oral plaque.

U.S. Pat. No. 5,747,005 discloses an oil-based, anti-plaque dentifrice comprising vitamin E and an enzyme in an admixture which is effective to dissolve plaque and to resist plaque formation. The preferred dentifrice composition is substantially anhydrous, and includes a chelating agent and a promoter to assist the enzyme in fighting plaque. The oil base is about 55 to 70 percent of the total composition derived from flavoring or taste-enhancing oils of wintergreen, spearmint, peppermint, cinnamon or clove.

U.S. Pat. No. 5,776,435 discloses an oral composition comprising in an orally acceptable aqueous solution, about 5-30% of a siliceous polishing agent, about 0.25%-0.35% of triclosan, at least one of a surface active agent and a flavoring oil, and about 0.05-4% of a copolymer of maleic acid or anhydride with methyl vinyl ether. U.S. Pat. No. 5,494,660 discloses an oral composition containing poloxamines. Poloxamines are claimed to inhibit adhesion of microbes to biological surfaces, including teeth and oral mucosa.

U.S. Pat. application No. 20050118115 discloses an improved composition for whitening of a tooth. The composition contains hypochlorite in combination with peroxide which reacts on the surface of the tooth being whitened. The hypochlorite may also contain a flavoring agent including mint, oil of wintergreen, oil of peppermint, oil of spearmint, clove bund oil, menthol, anethole, methyl salicylate, eucalyptol, cassia, 1-menthyl acetate, sage, eugenol, parsley oil, oxanone, alpha-irisone, marjoram, lemon, orange; propenyl guaethol, cinnamon, vanillin, thymol, linalool, cinnamaldehyde glycerol acetal, and mixtures thereof.

Although prior oral compositions of the type described above are generally suitable for application to teeth and gums by brushing, or rinsing such delivery means are insufficient for individuals subject to high plaque and calculus formation at dental implant sites. Dental implants have a different surface than natural teeth for accumulation of plaque. They are typically composed of three portions. First, there is titanium alloy fixture which is affixed to alevolar bone. Second, there is titanium alloy collar transmucsal abutment at the gingiva mucosa surface where plaque formation tends to be greatest. Third, there is a porcelain prosthesis which is attached to the collar. Effective removing of plaque and calculus from dental implants is therefore more challenging as compared with natural teeth.

Accordingly, there is continued interest in the development of new natural oral compositions that provide for adequate cleaning propertise while at the same time provide for improved comfort to the user without irritating and damaging the teeth, gums or the bone around one's teeth. The invention described herein addresses this and other needs by providing oral compositions that cause optimal improvement of gum and oral health by preventing and treatment of gum and oral health problems.

### SUMMARY OF THE INVENTION

The invention provides novel anti-calculus oral compositions comprising a substantially anhydrous plant oil mixture and orally acceptable additives and carriers. The plant oil mixture comprising a major oil constituent and a minor oil constituent in a proportion of at least 10/1 respectively, wherein the major oil constituent comprises an edible vegetable oil and the minor oil consitutent comprises an emulsifying oil, flavoring oil, antiseptic oil, anti-inflammatory oil, or a combination thereof.

In one embodiment, the major oil constituent comprises sunflower oil, and the minor oil constituent comprises citrus oil, mint oil, rosemary oil, chamomile oil, or clove oil, or a combination thereof. The concentration of the sunflower oil is in the amount of from about 80% to about 88% v/v, the citrus oil is from about 1% to about 3% v/v, the mint oil is from about 1% to about 2% v/v, the rosemary oil is from about 1% to about 2% v/v, the chamomile oil is from about 0% to about 4% and the clove oil is from about 1% to about 4% v/v.

In another embodiment, the anti-calculus oral composition further comprises eucalyptus oil, pine needle essential oil, zalby, or a combination thereof.

The anti-calculus oral composition of the invention can be made into variety of oral formulations including dry formulation, gel formulation, or an emulsion. The emulsion is water in oil or oil in water emulsion. The oral formulations include toothpaste, gel dentifrice, tooth powder, mouthrinse, mouthwash, tooth hardener, antiplaque composition, gum or lozenge.

In one embodiment, the anti-calculus oral composition further comprises an additive comprising cleaning agents, softening agents, surfactants, processing aids, coloring or pigment agents, astringents, antiseptics, stimulants, flavoring agents, abrasives for improved cleaning, dental desensitizer, dental whitener, antioxidants, chelators, or a combination thereof. The astringent comprises bayberry bark, white oak bark, rhatany bark, sage, and teas, or a combination thereof. The antiseptics comprise antibacterial agent, antiviral agent, antifungal agent, or a combination thereof and include peppermint oil, echinacea, bloodroot, cayenne, tea tree oil, wild bergamont, chaparral, stinging metal, bay, myrrh, rhatany bark, toothache tree, calendula, and chamomile, or a combination thereof, among others.

The flavoring agent comprises mint, oil of wintergreen, oil of peppermint, oil of spearmint, clove bund oil, menthol, anethole, methyl salicylate, eucalyptol, cassia, 1-menthyl acetate, sage, eugenol, parsley oil, oxanone, alpha-irisone, marjoram, lemon, orange, propenyl guaethol, cinnamon, vanillin, thymol, linalool, cinnamaldehyde glycerol acetal, and a combination thereof, among others.

In another aspect, the invention provides an oral hygiene kit comprising a composition containing an effective anti-calculus amount of a substantially anhydrous plant oil mixture. The plant oil mixture comprises a major oil constituent and a minor oil constituent in a proportion of at least 10/1 respectively, wherein the major oil constituent comprises an edible vegetable oil and the minor oil consitutent comprises emulsifying oil, flavoring oil, antiseptic oil, anti-inflammatory oil, or a combination thereof, and instructions to use the kit.

In one embodiment, the major oil constituent comprises sunflower oil and the minor oil constituent comprises citrus oil, mint oil, rosemary oil, chamomile oil, and clove oil, or a combination thereof. The oral hygiene kit can further comprise eucalyptus oil, pine needle essential oil, zalby, or a combination thereof.

In yet another aspect, the invention provides a method for preventing, treating, or ameliorating dental and gum disease comprising administrating to a patient in need thereof and effective amount of a composition comprising a substantially anhydrous plant oil mixture. The plant oil mixture comprising a major oil constituent and a minor oil constituent in a proportion of at least 10/1 respectively, wherein the major oil constituent comprises an edible vegetable oil and the minor oil consitutent comprises an emulsifying oil, flavoring oil, antiseptic oil, anti-inflammatory oil, or a combination thereof.

### DETAILED DESCRIPTION OF THE INVENTION

The invention as described herein provides new oral compositions effective for maintenance of oral health and provide for the prevention and treatment of gum and dental diseases. The invention uses a unique combination of ingredients which exhibit desired properties. The ingredients have demonstrated remarkable synergistic anti-calculus and anti-plaque effects. The composition of the invention can be used as an effective cleaner of interdental and interproximal spaces and as an interdental stimulator without a traumatic effect on the crest. In one embodiment, the composition is used to remove crystals and calculus from dental implants, denture, crowns, gums, and other dental prosthesis.

The composition of the invention is particularly useful for children and elderly, who are unable to floss or brush effectively, people with gum and or dental diseases, or people having prosthesis or dentures.

According to a embodiment of the invention, there is provided a method of cleaning the mouth, teeth and gums for cosmetic purposes by oral application of any oral hygiene composition as defined herein.

1. Anti-calculus Compositions

The compositions of the invention comprise an oil mixture as the active ingredient. The oil in the mixture comprises a wide variety of oils, including essentially plant derived oils, so long as the mixture of oil demonstrates anti-plaque, anti-calculus, antibacterial and/or anti-staining properties. In one embodiment, the compositions of the invention comprise a mixture of plant derived oil that demonstrates synergistic anti-calculus and/or anti-plaque properties. The plant derived oil includes essential oils, flavoring oils, edible or non-edible oils, carrier oils, or a combination thereof among others.

Also encompassed within the scope of the invention is the incorporation of one or more additives within a dosage range known by those of ordinary skill in the art to be safe for use in oral hygiene applications. The additives include active and/or non-active agents including, by way of example and not limitation, cleaning agents, softening agents, chelating agents, surfactants, processing aids, coloring or pigment agents (e.g., titanium dioxide to impart color to the teeth or prosthesis, among others), flavoring agents, sweetening agents (e.g., sucrose, saccharin, or sodium or calcium cyclamate, among others) abrasives for improved cleaning (e.g., kaolin, clay, and silica, among others) tooth desensitizing agents, tooth whitener, antioxidants (to prevent discoloration e.g., tocopherol, sodium metabisulphite, butylated hydroxytoluene (BHT), butylated hydroxyanisole (BHA), among others) ascorbic acid or sodium ascorbate.), antiseptics (e.g., antibacterial agents, antiviral agents, antifungal agents, or a combination thereof, among others), humectants, gelling agents, fluoride sources, sweeteners, preservatives (e.g., ethyl, n-propyl, or p-hydroxy benzoate, among others) structuring agents, anti-calculus agents, anti-plaque agents, astringents (e.g., bayberry bark, white oak bark, rhatany bark, sage, and teas, or a combination thereof, among others), and stimulants, or a combination thereof, among others.

The additive may be supplied in any clinically acceptable form or shape. For example, the additive can be mixed into the solution or emulsion, made into a microencapsulated form, or adsorbed or absorbed onto another additive, e.g., particulate filler. Flavoring, for example, can be released by the composition through incorporating a flavoring agent into the composition. The additive, if desired, can also be incorporated in encapsulated form.

Plant oils are the pure “essence” of a plant and have been found to provide biological benefits when used correctly and safely. There are more than about 90 different oils encompassed within the scope of the invention. The plant oil within the scope of the invention is a plant liquid or extract that is generally distilled (most frequently by steam or water) from the leaves, stems, flowers, bark, roots, or other elements of a plant. A majority of the plant oil used within the composition of the invention, contrary to the use of the word “oil” are not really oily-feeling at all. These oils are generally clear, but some oils such as patchouli, orange and lemongrass are amber or yellow.

Flavor oils are generally artificially created fragrances or contain artificial substances. In one embodiment the composition of the invention contain natural flavoring oils including eucalyptus oil, pine needle essential oil, zalby, or a combination thereof, among others. Citrus oil and mint oil change the texture of the composition from being oily to an emulsified solution. Carrier oils (e.g., sweet almond oil, apricot kernel oil, grapeseed oil, or a combination thereof, among others) may be used to dilute a composition.

The plant oil encompassed within the scope of the composition of the invention includes, by way of example and not limitation, sunflower oil, safflower oil, citrus oil, mint oil, rosemary oil, chamomile oil, clove oil, wintergreen oil, spearmint oil, peppermint oil, cinnamon oil, castor oil, tea oil, anise oil, basil oil, bay oil, beeswax oil, cardamom oil, carrot seed oil, cedarwood oil, citronella oil, sage oil, coriander oil, cypress oil, dill oil, elemi oil, eucalyptus oil, fennel oil, geranium oil, ginger oil, grapefruit oil, helichrysum oil, hyssop oil, immortelle oil, jasmine oil, juniper Berry oil, lavender oil, lemon oil, lemongrass oil, lime oil, linden blossom oil, mandarin oil, marjoram oil, myrrh oil, myrtle oil, neroli oil, nutmeg oil, oakmoss oil, orange oil, oregano oil, palmarosa oil, parsley oil, patchouli oil, pepper oil, peppermint oil, petitgrain oil, pine oil, ravensara oil, rose oil, rosemary oil, rosewood oil, sandalwood oil, spearmint oil, spikenard oil, spruce oil, tangerine oil, thyme oil, tuberose oil, vanilla oil, vetiver oil, violet leaf oil, yarrow oil, almond oil, apricot oil, avocado oil, canola oil, chile oil, coconut oil, sesame oil, corn oil, cotton seeds oil, flax seeds oil, olive oil, grape seeds oil, walnut oil, hazelnut oil, palm oil, mustard oil, peanut oil, pine seeds oil, poppy seeds oil, arachnid oil, rice oil, pumpkin oil, soybean oil, wheat germ oil, zalby or a combination thereof, among others.

To achieve the desired results, the preferred formulation exhibits astringent, antiseptic, local stimulant, anti-inflammatory, and local analgesic properties. These properties can be attained by using a combination of components, wherein each component exhibits one or more of the desired properties. The formulation can contain more than one component which exhibits the same desired property.

For ease of production, preferred components are in the form of a liquid, such as fluid extracts, tinctures, solid extracts, and the like, but can also be in the form of powders and the like. To promote health and ensure safety, it is also desirable that the components be extracts, oils, powders, etc., of naturally occurring flora and that these components are not chemically processed other than the process used to directly produce the fluid extract, tincture or oil, and are thereby suitable for use in the oral cavity.

Antiseptics encompassed within the scope of the invention are derived from variety of sources. In a preferred embodiment, the antiseptics are plant oil or plant extracts derived from plants such as, for example, peppermint, echinacea, bloodroot, cayenne, tea tree, wild bergamont, chaparral, stinging metal, bay, myrrh, rhatany bark, toothache tree, calendula, and chamomile, or a combination thereof.

Stimulants are anther type of additives that may be included in the composition of the invention. In a preferred embodiment, stimulants are plant oil or plant extracts derived from plants such as, for example, cayenne, peppermint, calendula, wintergreen, bayberry, and sage, or a combination thereof, among others.

In one embodiment, the composition of the invention additionally contains one or more active or inactive ingredients comprising a cationic antibacterial agent or an orally acceptable salt thereof, a water soluble anionic active agent or an orally acceptable salt thereof, a combination of a non-ionic and/or an amphoteric surfactant having a high hydrophilic/lipophilic balance. The combination of surfactants facilitates stabilization of the composition, thus ensuring effective antimicrobial action and long shelf life.

The antibacterial agent is present in an effective amount, typically from about 0.01-5% w/w, from about 0.01-0.6% w/w, or from about 0.03-0.3% w/w. The antibacterial agent is substantially water-insoluble, which means that its solubility is less than about 0.1% to about 1% by weight in water at 25° C.

Examples of water insoluble antibacterial agents include, by way of example and not limitation, halogenated diphenyl ethers (e.g., 2′,4,4′-trichloro-2-hydroxy-diphenyl ether (Triclosan), 2,2′-dihydroxy-5,5′-dibromo-diphenyl ether), halogenated alicylanilides (e.g., 4′,5-dibromosalicylanilide, 3,4′,5-trichlorosalcylanilide, 3,4′,5-ribromosalicylanilide, 2,3,3′,5-tetrachlorosalicylanilide, 3,3,3′,5-tetrachlorosalicylanilide, 3,5-dibromo-3′-trifluoromethyl salicylanilide, 5-n-octanoyl-3′-trifluoromethyl salicylanilide, 3,5-dibromo-4′-trifluoromethyl salicylanilide, 3,5-dibromo-3′-trifluoro methyl salicylanilide (Fluorophene)), benzoic esters (e.g., methyl-p-hydroxybenzoic ester, ethyl-p-hydroxybenzoic ester, propyl-p-hydroxybenzoic ester, butyl-p-hydroxybenzoic ester, halogenated carbanilides, 3,4,4′-trichlorocarbanilide, 3-trifluoromethyl-4,4′-dichlorocarbanilide, 3,3,4′-trichlorocarbanilide), phenolic Compounds (e.g., phenol and its homologs, mono- and poly-alkyl and aromatic halo such as F, Cl, Br, I)-phenols, resorcinol and catechol and their derivatives and bisphenolic compounds). Such compounds include inter alia: phenol and its homologs, phenol, 2 methyl-phenol, 3 methyl-phenol, 4 methyl-phenol, 4 ethyl-phenol, 2,4-dimethyl-phenol, 2,5-dimethyl-phenol, 3,4-dimethyl-phenol, 2,6-dimethyl-phenol, 4-n propyl-phenol, 4-n-butyl-phenol, 4-n-amyl-phenol, 4-tert-amyl-phenol, 4-n-hexyl-phenol, 4-n-heptyl-phenol, 2-methoxy-4-(2-propenyl)-phenol (Eugenol), 2-isopropyl-5-methyl-phenol (Thymol), mono- and poly-alkyl and aralkyl halophenols, methyl-p-chlorophenol, ethyl-p-chlorphenol, n-propyl-p-chlorophenol, n-butyl-p-chlorophenol, n-amyl-p-hlorophenol, amyl-p-chlorophenol, 3-methyl-p-chlorophenol, 3,5-dimethyl-p-chlorophenol, 6-ethyl-3-methyl-p-chlorophenol, 6-n-propyl-3-methyl-p-chlorophenol, 6-iso-propyl-3-methyl-p-chlorophenol, 2-ethyl-3,5-dimethyl-p-chlorophenol, 6-sec butyl-3-methyl-p-chlorophenol, resorcinol and its derivatives, 2-iso-propyl-3,5-dimethyl-p-chlorophenol, p-bromophenol, phenyl phenol, methylphenol, 5-chloro-2-hydroxydiphenyl methane, famesol, nerolidol, bisabolol, thymol, eugenol, and 2,2′methylene bis(4-chloro-6-bromophenol), or a combination thereof, among others.

The antibacterial agents may be combined with one or more mineral anti-calculus agents such as zinc ions, copper compounds, potassium ions, or a combination thereof.

In another embodiment, the composition additionally contains an antibacterial-enhancing agent which enhances delivery of the antibacterial agent and its retention on oral surface. The antibacterial enhancing agents comprise polymeric materials including materials that have been characterized as having utility as dentifrice adhesives or fixatives or dental cements. For example, commercially available copolymer of methylvinyl ether-maleic anhydride (Gantrez) as a denture fixative. The adhesives, fixatives or cements when applied in water-soluble or water-swellable form together with substantially water-insoluble non-cationic antibacterial antiplaque agents enhance the antibacterial activity of such agents. See, for example, U.S. Pat. No. 4,485,090 disclosing polymeric anionic membrane-forming materials that attach to a tooth surface to form a hydrophobic barrier which reduces the formation of plaque. Such polymeric material could enhance the antibacterial activity of substantially water-insoluble anti-calculus oil composition of the invention.

The antibacterial enhancing agents may be a simple natural or synthetic compound or a polymer that is water insoluble or water (saliva) soluble or swellable (hydratable, hydrogel forming). The polymer includes, for example, oligomers, homopolymers, or copolymers of two or more monomers, among others. The antibacterial enhancing agent is employed in amounts effective to achieve such enhancement within the range of about 0.01% to about 1%, about 0.3% to about 2%, or about 3% to about 4% by weight.

In one embodiment, the composition of the invention contains an additive such as a gel component that imparts softness and and viscosity. The gel component includes a variety of materials, including for example, at least one styrenic based elastomeric polymer, in combination with at least one oil plasticizer or flexibilizer, such as a mineral oil, silicone oil, naphthenic oil, parafinic oil and the like. The ratio of elastomeric polymer to oil in the gels typically ranges, for example, from about 100 parts polymer to about 100, 200, 300, 400, 1000, 2000 parts or more oil.

Styrenic based polymers will typically be copolymers of styrene and one or more monomers, usually olefinic monomers, where illustrative olefinic monomers include ethylene, propylene, butylene, and the like. Specific styrenic based copolymers of interest include, for example, (styrene-ethylene-propylene-styrene) copolymers, such as those sold under the trade names SEPTON 2006, SEPTON 4055, and the like.

Oil useful in the gel component of the composition of the invention include, by way of example and not limitation, high, medium and low viscosity oils including, for example, oil sold under the trade names Sentry Dimethicone NF 350, and the like, mineral oil including, for example, and oil sold under the trade names Duoprime, Kaydol, Hydrobrite, Britol, and the like.

The composition of the invention may additionally contain a surfactant. Suitable surfactants are water-soluble organic compounds, and may be nonionic, cationic or amphoteric species. The surfactant used should preferably be stable, able to form suds throughout a wide pH range, and able to produce a foam in use.

Suitable nonionic surfactants include the products of the condensation of alkylene oxide groups with aliphatic or alkylaromatic species, and include, for example, polyethylene oxide condensates of alkyl phenols, ethylene oxide/propylene oxide copolymers (such as those available from BASF Wyandotte Chemical Corporation under the trade name ‘Pluronic’) ethylene oxide/ethylene diamine copolymers, ethylene oxide condensates of aliphatic alcohols, long chain tertiary amine oxides, long chain tertiary phosphine oxides, long chain dialkyl sulfoxides and mixtures thereof. Alternatives include ethoxylated sorbitan esters such as those available from ICI under the trade name “Tween”.

Cationic surfactants are generally quaternary ammonium compounds having one C₈₋₁₈ alkyl chain and include, for example, lauryl trimethylammonium chloride, cetyl trimethylammonium bromide, di-isobutylphenoxy-ethoxyethyldimethyl-benzylammonium chloride, coconutalkyltrimethylammonium nitrite, cetyl pyridinium fluoride. Also useful as cationic surfactants are benzyl ammonium chloride, benzyl dimethyl stearylammonium chloride, and tertiary amines having one C₁₋₁₈ hydrocarbon group and two polyoxyethylene groups.

Amphoteric surfactants are generally aliphatic secondary and tertiary amines comprising aliphatic species which may be branched or unbranched, and in which one of the aliphatic species is a C₈₋₁₈ species and the other contains an anionic hydrophilic group, for example, sulfonate, carboxylate, sulfate, phosphonate or phosphate. Examples of quaternary ammonium compounds are the quaternized imidazole derivatives available under the trade name ‘Miranol’ from the Miranol Chemical Company.

Also encompassed within the scope of the composition of the invention is the use of a structuring agent. Structuring agents may be useful in, for example, dentifrices and gums to provide desirable textural properties and “mouthfeel”. Suitable agents include natural gum binders such as gum tragacanth, xanthan gum, gum karaya and gum arabic, seaweed derivatives such as Irish moss and alginates, smectite clays such as bentonite or hectorite, carboxyvinyl polymers and water-soluble cellulose derivatives such as hydroxyethyl cellulose and sodium carboxymethyl cellulose. Improved texture may also be achieved, for example, by including colloidal magnesium aluminum silicate. Suitably, the structuring agent is included in an amount of from 0-3%, preferably 0-0.1% by weight of the oral hygiene composition.

The composition of the invention may also include an abrasive agent. Abrasives should preferably be capable of cleaning and/or polishing the teeth without causing harm to dental enamel or dentine. Suitable abrasives include the silica abrasives, such as hydrated silica and silica gels, particularly silica xerogels such as those available under the trade name ‘Syloid’ from W. R. Grace and Company. Also suitable are precipitated silica materials such as those available under the trade name ‘Zeodent’ from J. M. Huber Corporation, and diatomaceous earths such as those available under the trade name ‘Celite’ from Johns-Manville Corporation. Alternative abrasives include alumina, insoluble metaphosphates such as insoluble sodium metaphosphate, calcium carbonate, dicalcium phosphate (in dihydrcate and anhydrous forms), calcium pyrophosphate (including beta-phase calcium) polymethoxylates and particulate thermosetting polymerised resins such as, for example, ureas, melamine-ureas, melamine-formaldehydes, urea-formaldehydes, melamine-urea-formaldehydes, cross-linked epoxides, melamines, phenolics, and cross-linked polyesters.

In one embodiment, a fluoride source is included in the composition of the invention. Inclusion of a fluoride source in the composition is beneficial, since fluoride ions are known to become incorporated into the hydroxyapatite of tooth enamel, thereby increasing the resistance of the enamel to decay. Inclusion of a fluoride source is also desirable when a polyphosphate anti-calculus agent is included, in order to inhibit the enzymic hydrolysis of such polyphosphates by salivary phosphatase enzymes. Fluoride sources suitable for use in oral hygiene compositions of the present invention include sodium fluoride, zinc fluoride, potassium fluoride, aluminum fluoride, lithium fluoride, sodium monofluorophosphate, acidulated phosphate fluoride, stannous fluoride, ammonium fluoride, ammonium bifluoride and amine fluoride. Preferably, the fluoride source is present in an amount sufficient to provide from about 50 ppm to about 4,000 ppm fluoride ions in use.

Also encompassed within the scope of the invention is the use of a desensitizing agent. Suitable desensitizing agents include, by way of example and not limitation, formaldehyde, potassium nitrate, tripotassium citrate, potassium chloride and strontium chloride (suitably as hexahydrate), strontium acetate (suitably as hemihydrate) and sodium citrate/Pluronic (Trade Name; see above) gel.

Flavoring agents may be added to increase palatability and may include, for example, oil of peppermint, spearmint, wintergreen, sassafras and clove. Sweetening agents may also be used, and these include D-tryptophan, saccharin, dextrose, aspartame, levulose, acesulfam, dihydrochalcones and sodium cyclamate. Typically, such flavoring and sweetening agents are included in amounts of from about 0-5%, preferably from about 0-2% by weight of the oral hygiene composition.

Coloring agents and pigments may be added to improve the visual appeal of the composition. Suitable colorants include dyes such as, for example, FD & C blue No. 1, D & C yellow No. 10 and D & C yellow No. 3. A suitable and commonly used pigment is titanium dioxide, which provides a strong white color.

It is contemplated that additional components may include coloring and flavoring additives having multi-property agents. As an example of multi-property agent is peppermint oil that takes advantage of the local stimulant and antiseptic properties as well as providing a flavoring component.

To promote health and ensure safety, it is also desirable that the active and non-active ingredients of the anti-calculus composition of the invention have been extracted from naturally occurring flora and that these components are not chemically processed other than the process used to directly produce the fluid extract, tincture or oil, and are thereby suitable for use in the oral cavity.

In one embodiment, the composition contains an edible oil such as, for example, the sunflower oil, ant least two or at least three other plant oils comprising citrus oil, mint oil, rosemary oil, chamomile oil, clove oil, or a combination thereof.

Sunflower oil (Helianthus annuus) contains Linoleic acid (57%) and linolenic acid (33%) and a small fraction of palmitin, acetadin, arashedin, and linocerin. The oil extracted from its kernels contains unsaturated fatty acids such as ac. Heliantannic, ac. Heliantice, and ac. solanttic, which penetrate and dissolve microbial plaque accumulation on the teeth.

Citrus lemon oil contains limonene along with phellandrene, camphene, and pinene. The fragrant of citrus lemon essence pertains to citral. Citrus lemon essence contains acid citric, calcium potassium citrate, and limonene (C₁₀H₁₆), along with free/neutral geraniol, linalool, citronellol, aldehydes nonelitic, and acid antrnilic. Therapeutic properties of citrus lemon oil include, anti-scorbutic, antiseptic, disinfection, as well as ability to dissolve the sediments and deposits on the teeth to make it bright and strengthening the gums. The citrus lemon has powerful antiseptic properties by neutralizing the bacterial growth (e.g., Meningocoque, Eberthbacilli of typhoil, Pneumococcus of Pneumonia, Staphylococcus dor, Streptococci hemolytic, and Leoflerbacilli diphtheria) in the mouth cavity within 5-20 minutes after administration.

Mint oil (Mintha aronesis) contains menthol essence, mentol ethers (e.g., acetate, butyrate, and isocarbonate of methone such as, for example, menthene fatty acids, free valornic acids and acetic acids. Mentol essence is used for disinfecting, refreshing and cooling the tongue surface.

Rosemary oil (Rosmarinus officianlis) contains the essence of Romarin, which is aromatic and yellow in color, insoluble in water, but soluble with ten times volume of alcohol (80%) or a half volume of alcohol (95%). Additionally, it contains Borneol, acetatae iso-bornyle, camphene, pinene, and small quantity of cinoeole. This oil is used externally, for example in an antiseptic gargle, to prevent coated (flurred) tongue, dental tonsillitis, removing plaque and deposits and refreshing the mouth cavity.

Chamomile oil (Matricaria chamomilla). The sepals of this plant yields about 20-80% of a dark blue essence when fresh due to Azulene, a powerful anti-inflammatory pheleojestic and healing agent, along with sesquiterpenes: B, C, and a sesquiterxniques alcohol type such as comomillo, caprique, nonylique of the alcohol, furfurol and fatty acids. Additionally, matricarine (C₁₇H₂O O₅) molecular weight 337.36 KDa, anthemic acid, anthemidine, and Taten are extracted from this flower. Chamomile oil has a strong anti-inflammatory activity and it is used for the treatment of mouth ulcer, disinfecting gums, teeth, tongue and soft issues. The anti-microbial activities of azulene is pronounced in week solutions of chamomile (e.g., solution of 1/200 dilutions), which is effective against Staphylococcus dor, Streptococci hemolytic.

Clove oil (Oleum caryophyllic) contains as principal components eugenol, acid eugenic, acid caryophyllic, and ally-guaiacol (C₁₀H₁₂ O₂) molecular weight of 164.20 KDa. Tanen is a sticky substance that contains about 13% of a colorless and a crystalline compound of caryophyllen. Eugeno is especially used in dentistry as sedative, toothache pain reliever, and anti-inflammatory. Clove oil has also disinfectant properties and can help prevent formation of microbial plaque on the teeth.

An exemplary formulation of the anti-calculus composition of the invention comprises:

| | |
|---|---|
| 1. Sunflower oil (Helianthus annuus) | 80%-88%% by volume |
| 2. Citrus oil(citrus lemon essence) | 1%-3% by volume |
| 3. Mint oil (Mintha aronesis) | 1%-2% by volume |
| 4. Rosemary oil (Rosemarinus officinalis) | 1%-2% by volume |
| 5. Chamomile oil (Matricaria chamomilla) | 0%-1% by volume |
| 6. Clove oil (Oleum caryophylli) | 1%-4% by volume |

Another exemplary formulation of the anti-calculus composition of the invention includes one or more of the following essences:

| | |
|---|---|
| 7. Eucalyptus oil | about one drop per about 150 ml |
| 8. Pine needle essential oil (Pinus | about one drop per about 150 ml |
| silvestris) | |
| 9. Zalby (Zallua officinalis) | about one drop per about 150 ml |

Eucalyptus oil, pine needle oil, and zalby are available as an essence. Eucalyptus oil helps in preventing inflammation and infection of the gum and tissues that surround the teeth and also assist in removing the rancid mucous stuck at the far end of the tongue. The purity of Eucalyptus oil is about 88% or more.

2. Formulations

The composition of the invention is formulated into several oral hygiene formulations which provide optimal treatment of gum and oral diseases and provide for the prevention of gum and oral health problems. The composition of the invention uses a unique combination of ingredients which exhibit desired properties including antiseptic, astringent, stimulant, local analgesic properties, or a combination thereof, which in the present formulations result in the synergistic properties of all the components.

The compositions of the invention are in the form of sterile, pyrogen-free preparations for example as a sterile substantially non-aqueous solution, a suspension or an emulsion. As used herein, substantial non-aqueous solution, or substantial anhydrous solution refers to a solution that contains at least 80% oil.

The composition formulations may conveniently be prepared by conventional techniques. Such techniques include the step of bringing into association the active ingredient and the carrier(s) or excipient(s). In general, the formulations are prepared by uniformly and intimately bringing into association the active ingredient with liquid carriers or finely divided solid carriers or both, and then, if necessary, shaping the product.

The plant oil mixture and other constituents of the composition of the invention may be formulated into a single or a separate composition. Thus, in one embodiment, the invention provides a single dental composition comprising the oil mixture. Those constituents that are water soluble, are generally not formulated into a single dental composition with the oil mixture, but are rather formulated as separate compositions.

In a preferred embodiment, the water soluble constituents, the plant oil mixtures, and the remaining ingredients are formulated into a single dental composition as an emulsion, for example an oil-in-water emulsion or water-in-oil emulsion.

The above formulations result in a concentrate or a diluted from which can be used in any number of application methods, formulations and compositions. The concentration of the product can be varied to achieve desired results. The composition of the invention is formulated in a diluted or a concentrate form for use in any form of interdental or periodontal treatment and/or prevention. The formulations include, for example, toothpaste, oral gel, poultice (e.g., dental dressing) paste, mouth wash, spray, dentifrice, toothpowder, chewing gum, lozenge, or impregnated floss or brushes, among others.

In one embodiment, the concentrated composition is diluted with a purified liquid such as, for example, water, alcohol, extracts, oil, etc. and used as a mouthwash. A preferred rinse comprises up to about 5% to about 10% or more by volume of the concentrate diluted with the purified liquid. The concentrate could be impregnated in or coated on a dental floss or fibers of a toothbrush. The concentrate can also be added to a toothpaste base, oral gel, or other oral compositions.

The concentration of the components of the oil mixture can be varied in these formulations depending on the specific route of administration and the end result sought. Accordingly, the composition is prepared in several concentration forms. A diluted solution is used as an oral irrigator or mouth wash which bolsters maximum penetration into gum pockets. The concentrate can be formulated into toothpaste, oral gel, poultice (e.g., dental dressing) paste, and the like. In another embodiment, the solution is made into an oral gel composition in an orally acceptable gel vehicle. The oral gel may be applied by a dental syringe.

It should be understood that in addition to the compositions, particularly mentioned above, the formulations of the present invention may include other agents conventional in the art having regard to the type of formulation in question. The composition of the invention includes compositions for human and veterinary dental hygiene use.

3. Administration and Dosage

The dosage of the compositions of the present invention will depend on a specific dental and/or gum disease state and other clinical factors such as age, and physical and condition of the human or animal and the route of administration of the compounds or compositions. The precise dose to be employed in the formulation, therefore, should be decided according to the judgment of the dental care practitioner and each patient's circumstances. Effective doses may be extrapolated from dose-response curves derived from in vitro tests, in vivo tests, animal model test systems, or clinical trials.

An “effective amount” of the compositions of the invention is any amount sufficient to therapeutically inhibit the progression of a dental and/or gum disease or to prophylactically delay the onset of the disease symptoms. For example, the concentration of the plant oil mixture in a composition can range from about 60-99% or more, from about 65% to about 95% or more, from about 70% to about 90% or more, from about 75% to about 85% or more. It is intended herein that by recitation of such specified ranges, the ranges recited also include all those specific integer amounts between the recited ranges. For example, in the range of about 75% to about 85% or more, it is intended to also encompass 76%, 77%, 78%, 79%, 81%, 82% etc, without actually reciting each specific range therewith.

One of ordinary skill in the art can readily determine an appropriate temporal and interval regimen for administering the compositions of the invention. For example, the compositions of the invention can be administered once, twice or more daily, for one, two, three, four, five, six or seven days in a given a week. The length of time that the subject receives the composition can be determined by the subject's dentist or other health care providers and caretakers, according to need.

The compositions of the invention may be administered as a single treatment, or repeated treatments, over a period up to and including one week to about 48 months or more.

In one embodiment of the invention, the composition is administered to a subject, for example two to three times daily, for consecutive or non-consecutive days in a given week. In another embodiment, the composition is applied, for example once or twice, to the dental prosthesis of a subject daily.

In another embodiment, the composition is applied directly, or with some suitable carrier, to dental prosthesis to break up and remove plaque, firming the gum, providing sweet smelling mouth, and easy breathing. For example, a patient's prosthesis or denture can be immersed in the composition of the invention for a specific time of from about 1-12, or 12-24 hours or more, or about 1, 2, 3, 4, 5, 6, 7, 8, 9, 10 days or more depending on the concentration and the amount of calculus and/or plaques to be dissolved. The immersion of the prosthesis to the solution can be on a continuous or a discontinuous basis. For example, prosthetic cleaning may be conducted for a period of 24-48 hours, continuously or discontinuously having a time interval of about 2, 4, 6, 8, 10, or 12 hours or more immersion in a 24 hours period.

Additionally, the composition can be applied in 1-2 drops directly on a toothbrush or on commercially available toothpaste before brushing the teeth and gums of patients.

In addition, the compositions of the invention may be incorporated into biodegradable polymers allowing for sustained release of the composition, the polymers being implanted in the vicinity of where the delivery is desired, so that the composition is slowly released into the oral cavity.

4. Dental Hygiene Kits

Also encompassed within the scope of the invention are dental hygiene kits that contain the composition of the invention and optionally one or more dental hygiene instruments such as floss or toothbrush. The kit of the invention may also include one or more additives, among other solutions that may be used in the administration of the composition of the invention. The kit may be hermetically sealed to prevent tampering and the escape of any liquids or vapors from the package. The dental composition is subject to wide variation so long as it contains, as its primary constituents, the plant oil mixture of the invention.

The dental hygiene composition kit comprises one or more containers filled with one or more oil mixtures containing essential and/or non-essential fatty acids in a pre-determined ratio for mixing together. In one embodiment, the kit comprises instructions for preparing and using the dental hygiene composition and one or more of the following components: a plant oil composition, an additive, e.g., flavors, colors, antibacterial agents, and preservatives, astringents, stimulants, local analgesic, or a combination thereof.

Generally, the ingredients are supplied either separately or mixed together in unit dosage form, for example, as a dry lyophilized powder or water free concentrate in a hermetically sealed container such as an ampoule or sachets indicating the quantity of one or more active agents. If a particular component is not included in the kit, the kit can optionally comprise information on where to obtain the missing component, for example an order form or uniform resource locator for the internet specifying a website where the component can be obtained.

The instructions provided with the kit describe the practice of the composition of the invention as described above, and the route of administration and effective concentration and the dosing regimen for each of the compositions provided therein.

The composition may have ingredients added as necessary to provide a pleasant taste and odor, including for example, thyme, eucalyptol, menthol, and/or benzoic acid, among others. The composition may additionally contain antibacterial compositions tending to prevent the growth of deleterious bacteria contributing to the formation of plaque, tartar, halitosis, or the like. Instructions for use of the dental kit may be included or may be imprinted on the kit itself.

If a particular component is not included in the dental hygiene kit, the kit can optionally comprise information on where to obtain the missing component, for example an order form or uniform resource locator for the internet specifying a website where the component can be obtained.

This invention is illustrated by aforementioned description and examples, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims. The contents of all references, patents and published patent applications cited throughout this application are expressly incorporated herein by reference.

### EXAMPLES

### Example 1

### Anticalculus Efficacy of Three Oral Compositions

### PURPOSE: To evaluate the anticalculus effect of three anticalculus compositions of the invention.

MATERIALS AND METHODS: A 12-week clinical study is conducted on a population of calculus-forming adult male and female subjects in Germany and the results are generated for the anticalculus efficacy of three anticalculus compositions of the invention, as compared to a placebo dentifrice and a commercial dentifrice. The three inventive anticalculus compositions dentifrice are as follows: (1) A composition containing containing sunflower oil 80%, citrus oil 1%, Rosemary oil 1%, chamomile oil 1%, and clove oil 4%, (2) A composition containing sunflower oil 88%, mint oil 2%, rosemary oil 3%, and clove oil 1%, (3) A composition containing sunflower oil 85%, citrus oil 2%, rosemary oil 2%, and clove oil 4%, (triclosan and 0.75% zinc citrate in a 1.14% sodium monofluorophosphate/silica base. Placebo: 0.243% sodium fluoride/silica placebo
RESULTS: All three anticalculus compositions provide statistically significant reductions in supragingival calculus formation, as compared to a placebo dentifrice and the commercial dentifrice, after 12 weeks of use. The reductions in supragingival calculus formation ranged from 39% to 55%, as compared to a placebo dentifrice, for the three anticalculus compositions of the invention. There is no statistically significant difference among the three anticalculus compositions with regard to anticalculus efficacy.

### Example 2

### Efficacy of the Mouth-Rinse Formulation in Inhibiting the Development of Supragingival Dental Calculus

METHODS. After undergoing a dental prophylaxis, about 100 subjects with a moderate rate of calculus formation were stratified and randomly assigned to one of three groups: positive control (using a commercial toothpaste containing pyrophosphate and triclosan as an antibacterial agent and an antiseptic rinse), negative control (using placebo 0.243% sodium fluoride/silica) or experimental (using the mouth rinse of the composition 3 of example 1 above. Subjects brushed and rinsed twice daily, unsupervised, for four months. The researchers assessed subjects' severity and occurrence of supragingival calculus levels using the Volpe-Manhold Index, or VMI, after 16 weeks. The study design included a pre-test period where the calculus formation rate was measured in subjects brushing with a placebo
RESULTS. Using analysis of covariance, it is determined that the experimental group (which used the composition of the invention) and the positive control group (which used an anticalculus dentifrice containing pyrophosphate and triclosan) demonstrated statistically significantly lower VMI scores (P=0.001) than the negative control group (which used a regular dentifrice and an antiseptic rinse). Both anticalculus agents provided a clinically relevant 21 percent reduction in calculus formation. Conclusion. An antiseptic mouthrinse containing sunflower oil in the amount of 80% to 88% as the anticalculus agent provides a clinically relevant reduction in calculus formation in people with a moderate rate of such formation.

### Example 3

### In Vitro Results of Crystal Growth Inhibition and Plaque Biofilm Calcification

Laboratory studies were conducted to confirm the anti-calculus potential of the oral hygiene composition of the invention. The composition contains sunflower oil 85%, citrus oil 3%, mint oil 1%, rosemary oil 1%, chamomile oil 1%, clove oil 4%, and eucalyptus oil about one drop per about 150 ml.

RESULTS: Calcium hydroxyapatite crystal growth following direct supernatant treatments is significantly inhibited by the advanced composition of the invention. Similarly, plaque biofilm calcification was significantly inhibited by supernatant treatments with the composition. These results provide support for the anti-calculus and anti-plaque properties of the composition of the invention.

### Example 4

### Random Clinical Experiments

Experiment 1 A person having periodontal pockets in excess of 13 mm (almost ½ inch) deep had been recommended for treatment by surgery. In lieu of surgery the person began orally rinsing, twice-a-day, with the oral rinse of example 3. After four months of rinsing the periodontal pockets were reduced to about 6 mm and consequently were further reduced to about 4-3 mm after continuously using the mouth wash for another 6 months. After this period, the periodontal pockets were significantly reduced. Treatment also included daily brushing and flossing. After the six months of rinsing, no surgery was necessary.

Experiment 2 A person having periodontal pockets in excess of 5-7 mm deep had been recommended for extensive scaling and planing of the teeth. In lieu of scaling and planing the person began rinsing daily with the oral rinse from example 2. After 3 months of rinsing the periodontal pockets were reduced to a point where no scaling or planing was needed.

Experiment 3 A person rinsing daily with the oral rinse of Example 2 for a period of 4 years showed no signs of developing carries (cavities).

It will be understood by one of ordinary skill in the relevant arts that other suitable modifications and adaptations to the methods and applications described herein are readily apparent from the description of the invention contained herein in view of information known to the ordinarily skilled artisan, and may be made without departing from the scope of the invention or any embodiment thereof. All references discussed herein are incorporated by reference.

What is claimed is:

1. An anti-calculus oral composition comprising a substantially anhydrous plant oil mixture, the plant oil mixture comprising a major oil constituent and a minor oil constituent in a proportion of at least 10/1 respectively, wherein the major oil constituent comprises an edible vegetable oil and the minor oil consitutent comprises an emulsifying oil, flavoring oil, antiseptic oil, anti-inflammatory oil, or a combination thereof.

2. The anti-calculus oral composition of claim 1, wherein the major oil constituent comprises sunflower oil, and the minor oil constituent comprises citrus oil, mint oil, rosemary oil, chamomile oil, or clove oil, or a combination thereof.

3. The anti-calculus oral composition of claim 2, wherein the sunflower oil is in the amount of from about 80% to about 88% v/v, the citrus oil is from about 1% to about 3% v/v, the mint oil is from about 1% to about 2% v/v, the rosemary oil is from about 1% to about 2% v/v, the chamomile oil is from about 0% to about 4% and the clove oil is from about 1% to about 4% v/v.

4. The anti-calculus oral composition of claim 2, further comprising eucalyptus oil, pine needle essential oil, zalby, or a combination thereof.

5. The anti-calculus oral composition of claim 1, wherein the composition is a toothpaste, gel dentifrice, tooth powder, mouthrinse, mouthwash, tooth hardener, antiplaque composition, gum or lozenge.

6. The anti-calculus oral composition of claim 1, further comprising an additive comprising cleaning agents, softening agents, surfactants, processing aids, coloring or pigment agents, astringents, antiseptics, stimulants, flavoring agents, abrasives for improved cleaning, dental desensitizer, dental whitener, antioxidants, or a combination thereof.

7. The anti-calculus oral composition of claim 6, wherein the astringent comprises bayberry bark, white oak bark, rhatany bark, sage, and teas, or a combination thereof.

8. The anti-calculus oral composition of claim 7, wherein the antiseptics comprise antibacterial agent, antiviral agent, antifungal agent, or a combination thereof.

9. The anti-calculus oral composition of claim 8, wherein the antiseptics comprise peppermint oil, echinacea, bloodroot, cayenne, tea tree oil, wild bergamont, chaparral, stinging metal, bay, myrrh, rhatany bark, toothache tree, calendula, and chamomile, or a combination thereof.

10. The anti-calculus oral composition of claim 1, wherein the composition is in a dry formulation, gel formulation, or an emulsion.

11. The anti-calculus composition of claim 10, wherein the emulsion is water in oil or oil in water emulsion.

12. The anti-calculus composition of claim 6, wherein the flavoring agent comprises mint, oil of wintergreen, oil of peppermint, oil of spearmint, clove bund oil, menthol, anethole, methyl salicylate, eucalyptol, cassia, 1-menthyl acetate, sage, eugenol, parsley oil, oxanone, alpha-irisone, marjoram, lemon, orange, propenyl guaethol, cinnamon, vanillin, thymol, linalool, cinnamaldehyde glycerol acetal, and mixtures thereof.

13. An oral hygiene kit comprising a composition containing an effective anti-calculus amount of a substantially anhydrous plant oil mixture, the plant oil mixture comprises a major oil constituent and a minor oil constituent in a proportion of at least 10/1 respectively, wherein the major oil constituent comprises an edible vegetable oil and the minor oil consitutent comprises emulsifying oil, flavoring oil, antiseptic oil, anti-inflammatory oil, or a combination thereof, and instructions to use the kit.

14. The oral hygiene kit of claim 13, wherein the major oil constituent comprises sunflower oil, and the minor oil constituent comprises citrus oil, mint oil, rosemary oil, chamomile oil, and clove oil, or a combination thereof.

15. The oral hygiene kit of claim 14, wherein the sunflower oil is in the amount of from about 80% to about 88% v/v, the citrus oil is from about 1% to about 3% v/v, the mint oil is from about 1% to about 2% v/v, the rosemary oil is from about 1% to about 2% v/v, the chamomile oil is from about 0% to about 4% and the clove oil is from about 1% to about 4% v/v.

16. The oral hygiene kit of claim 15, further comprising eucalyptus oil, pine needle essential oil, zalby, or a combination thereof.

17. The oral hygiene kit of claim 15, wherein the composition is a toothpaste, gel dentifrice, tooth powder, mouthrinse, mouthwash, tooth hardener, antiplaque composition, gum or lozenge, or a combination thereof.

18. The oral hygiene kit of claim 13, further comprising an additive comprising cleaning agents, softening agents, surfactants, processing aids, coloring or pigment agents, astringents, antiseptics, stimulants, flavoring agents, abrasives for improved cleaning, dental desensitizer, dental whitener, antioxidants, chelators, or a combination thereof.

19. A method for preventing, treating, or ameliorating dental and gum disease comprising administrating to a patient in need thereof and effective amount of a composition comprising a substantially anhydrous plant oil mixture, the plant oil mixture comprising a major oil constituent and a minor oil constituent in a proportion of at least 10/1 respectively, wherein the major oil constituent comprises an edible vegetable oil and the minor oil consitutent comprises an emulsifying oil, flavoring oil, antiseptic oil, anti-inflammatory oil, or a combination thereof.

20. The method of claim 19, wherein the major oil constituent comprises sunflower oil, and the minor oil constituent comprises citrus oil, mint oil, rosemary oil, chamomile oil, or clove oil, or a combination thereof.
